(19) United States
(12) Patent Application Publication
(10) Pub. No.: US 2020/0268770 A1
(43) Pub. Date: Aug. 27, 2020

(54) PHARMACEUTICAL COMPOSITION USED FOR TREATING METABOLIC SYNDROME DISORDERS, INFECTIOUS DISEASES, AND COMPLICATIONS THEREOF

(71) Applicant: Guy Faustin MONKAM NITCHEU, Lille (FR)

(72) Inventor: Guy Faustin MONKAM NITCHEU, Lille (FR)

(21) Appl. No.: 16/481,853

(22) PCT Filed: Dec. 7, 2017
(86) PCT No.: PCT/IB2017/057731

§ 371 Date: Jul. 30, 2019

(30) Foreign Application Priority Data
Jan. 7, 2017 (FR) 17/70014
Nov. 7, 2017 (FR) 17/71175

(51) Int. Cl. A61K 31/575; A61K 31/045; A61K 31/11; A61K 31/12; A61K 31/353; A61K 45/06; A61K 31/015

(52) U.S. Cl. CPC A61K 31/575; A61K 31/045; A61K 31/11; A61K 31/015; A61K 31/353; A61K 45/06; A61K 31/12

(57) ABSTRACT

The present invention relates to a pharmaceutical composition, characterized in that it comprises, as active ingredient, a combination of d-limonene, lupeol and a pharmaceutically active agent selected from cinnamaldehyde, epicatechin, methylhydroxychalcone polymer, beta-sitosterol, curcumin and mixtures thereof. It is suitable for use in the preventive and curative treatment of obesity, diabetes, dyslipidaemias, infections caused by infectious agents and the consequences thereof, and in invasive cancers, in particular those associated with adipose tissues.

### TECHNICAL FIELD OF THE INVENTION

The present invention relates to a pharmaceutical composition that can be used as a medicament, in particular for the therapeutic treatment of concomitant or diabetic dyslipidemia following a pathology, cancers and in particular those associated with adipose tissue, neurodegenerative diseases, and for the treatment of diseases. infectious diseases, in this case HIV infection.

### Previous Art

The pandemics of obesity, metabolic syndrome and diabetes are directly associated with an incidence of early atherosclerosis and cardiovascular (heart, vascular) or arterial (arteries of the neck, legs, heart, etc.).

Although the pathogenesis of atherosclerosis is obviously multifactorial, dyslipidemia is an important predictor of cardiovascular risk in diabetics. In type 2 diabetes, dyslipidemia results in both quantitative and qualitative lipoprotein abnormalities (with small, dense, cholesterol-enriched LDL particles with particularly atherogenic properties, increased susceptibility to oxidation and their retention in the arterial wall), a moderate increase in triglycerides (TG), a lowering of HDL-cholesterol (HDL-c). In dyslipidemia of type 1 diabetic, quantitative abnormalities of lipoproteins are rare (except for type 1 diabetes with renal impairment). However, the qualitative alterations of lipoproteins are frequent and generally lead to an increase in the atherogenicity of LDL particles and a decrease in the anti-atherogenic power of HDL particles.

Most of the treatment data for dyslipidemia are from type 2 diabetic patients for whom dyslipidemia is highly correlated with insulin resistance and hyperinsulinemia. On the other hand, in patients with type 1 diabetes, the increased risk of cardiovascular disease should not be neglected and dyslipidemia treatments should also be proposed for these patients.

On the other hand, in HIV-positive patients treated with combinations of antiretrovirals, there are many harmful side effects, diabetes, lipodystrophy, dyslipidemia characterized by abnormal elevation of triglycerides and/or cholesterol. These infected patients are also at increased risk of developing atherosclerosis, this due to the negative effect of the Nef viral protein on the ABC AI transporter (ATP-binding cassette 1), a transporter involved in the efflux of cellular cholesterol. The cells of these patients acquire phenotypic changes. These cells (progenitor stem cells, adipocytes, glial cells, differentiated monocytes, macrophages, CD4+T lymphocytes, etc.) become more active, further storing lipid droplets, having a greater melting potential with viral particles, and act as a reservoir for the virus, inaccessible to antiretrovirals. Enrichment of these cells with lipids, particularly at the level of the cell membrane, could facilitate infection and viral replication. The membrane lipid rafts of these cells would play a major role.

These are membrane micro-domains, also called rafts, consisting of a compact assembly of lipids (glycosphingolipids and/or sphingomyelin and cholesterol) and proteins (glycosylphosphatidylinositol-anchored proteins, cholesterol-related proteins, transmembrane proteins doubly acetylated tyrosine kinases of the Src family, alpha subunits of heterotrimeric G proteins). Lipids acting as a solvent for membrane proteins. HIV (Human Immunodeficiency Virus) would interact with these lipid rafts both during its entry and during the budding of its virions, leading to the selective incorporation of certain lipids into the viral envelope and the rigidity of this virus. this. Some glycosphingolipids may even have an alternative cofactor or co-receptor function for HIV. Many more, as galactosylceramide would play an alternative receptor role in some HIV target cells.

This input mechanism using lipid rafts is also observed with some naked viruses or enveloped viruses such as measles virus, influenza virus, etc. The bacterium Escherichia Coli enters the mast cells by this route. Some pathogens (the prion protein, Plasmodium falciparum) use caveolae.

Caveolae represent a sub-category of these micro-domains. They contain a protein, caveolin linked to cholesterol and sphingomyelin.

In addition, GALT (Digestive Lymphocyte-Associated Tissue), the main reservoir for HIV, consists of 80% to 90% of the body's lymphocytes, the largest of which are memory and activated CD4+/CCR5 T cells. preferential targets of HIV-1. It is at the origin of numerous immunological abnormalities and architecture during infection with this virus. Alteration of this mucosa due to an early and massive depletion of CD4+T lymphocytes, particularly Th17 lymphocytes involved in mucosal immunity and its permeability, results in: bacterial translocation from the intestinal lumen to the blood circulation, disorders metabolic, systemic immune hyperactivation, chronic inflammation and amplification of viral replication. As a result, the progression of the disease and also the rapid development of non-infectious comorbidities such as, cardiovascular diseases, osteoporosis, cancers related or not to AIDS (acquired immunodeficiency syndrome), neurocognitive dysfunction, etc. In addition, adipose tissue is frequently found near invasive, aggressive cancers, particularly breast and prostate cancer. The mature adipocytes associated with cancer will then play a key role in tumor progression, the acquisition of a metastatic potential and in the resistance to chemotoxic treatments via the secretion of pro-inflammatory cytokines, proteins of the extracellular matrix and its remodeling, SDF-1 factor, etc. Some tumor cells overexpress glycosphingolipids, including globotriaosylceramide (Gb3), proteins such as the CXCR4 receptor, which is also an X4 tropism co-receptor. The establishment of the CXCR4/SDF-1 axis will then play a fundamental role in the constitution of an immunosuppressive microenvironment and the formation of a new blood supply. This dynamic interaction between adipocytes, lipid-loaded cells and tumor cells could be amplified under conditions of obesity and explain the poor prognosis observed in these patients with cancer.

On the other hand, dyslipidemias are also factors promoting neurodegenerative diseases, such as Alzheimer's disease, vascular dementia, etc. Indeed, in Alzheimer's disease, we observe in patients' brains, senile plaques consisting of amyloid peptide accumulation and neurofibrillary tangles characterized by the entanglement of a particular protein called tau protein. These senile plaques cause multiple cognitive deficits that would be partially corrected by action on the metabolism of cerebral cholesterol. The beta-amyloid peptide is involved in the reduction of LDL-cholesterol receptor membrane expression. This mechanism probably related to oxidative stress is similar to that observed in the reduction of the membrane expression of the insulin receptors and would explain the links observed between the Alzheimer's disease and insulin resistance. In addition, inflammatory responses of defenses caused by immune cells (macrophages, microglial cells, etc.) around these senile plaques would contribute to the progression of Alzheimer's disease.

Thus, in the management of dyslipidemias and the chronic inflammation that characterize a large number of diseases such as, neurodegenerative diseases, diabetes, glomerular nephropathies, cancer, AIDS, etc., hygienic measures Classical diets remain unavoidable, but only they are often insufficient and must therefore be supplemented by drug treatment.

Lipid-lowering therapies have emerged as potential weapons for reducing the risk of cardiovascular events in diabetic patients, those infected with HIV, and those with dyslipidemia. The therapeutic strategy consists of combining oral and/or injectable antidiabetic agents and/or lipid lowering agents. The combinations are variable depending on the patient's response to the therapy.

Oral antidiabetic drugs include: —Biguanides, they improve the sensitivity to insulin in the liver, muscles or fats. However, their action requires the presence of endogenous or exogenous insulin,
Hypoglycemic sulphonamides and glinides, they stimulate the secretion of insulin by beta cells of the pancreas. Their effectiveness depends on the residual capacity of the pancreas to secrete insulin, —The alpha-glucosidase inhibitors, they slow the passage of sugar from the food ingested from the intestine to the blood. He does not give hypoglycemia,
Incretins including GLP-1 (Glucagon-like peptide-1) are substances released by the body at the beginning of meals to stimulate the secretion of insulin. They are used in pharmacology either by injecting GLP-1 or by decreasing its degradation by the body thanks to the inhibitors of the enzyme DPP4 (dipeptidyl peptidase-4) or gliptins,
SGLT2 inhibitors (Sodium/glucose co-transporter type 2) increase the elimination of glucose in the urine.

Injectable forms include GLP-1 analogues, insulin (fast insulins, basal insulins and fixed insulin mixtures). Among the lipid-lowering agents, we find: —Statins, the most used, they are effective in lowering cholesterol levels in the blood, especially the LDL-cholesterol level. They reduce the risk of the occurrence or recurrence of diseases resulting from the narrowing or occlusion of arteries: myocardial infarction, angina pectoris, peripheral arterial occlusive disease, cerebrovascular accident,

The fibrates act via the receptor activated by the peroxisome proliferators of type a: PPARα. They reduce blood triglyceride levels and increase HDL-cholesterol “good cholesterol”. Their effectiveness in reducing the risk of developing cardiovascular diseases seems to be moderate, —Ezetimibe selectively inhibits the intestinal absorption of cholesterol and related phytosterols, —Nicotinic acid inhibits the release of free fatty acid from adipose tissue, which may contribute to decreased plasma levels of LDL-c, total cholesterol (CT), VLDL-c, apo B, triglycerides (TG), Lp (a) and an increase in HDL-c and Apo AI, all associated with a decrease in cardiovascular risk.
Omega 3 fatty acids are essential fatty acids necessary for the development and the good functioning of the human body, but that the body does not know how to manufacture.

However, data from recent literature have led to questions about the effect of statins on the risk of cardiovascular complications and the risk of disruption of glycemic control. Indeed, in the TNT study, diabetic patients receiving atorvastatin 80 mg had more cardiovascular events at the end of the study than non-diabetics receiving only atorvastatin 10 mg. This residual risk was also highlighted in the ACCORD-lipids study, particularly in secondary prevention and for patients with atherogenic dyslipidemia (characterized by an increase in TG and low HDL-c). On the other hand, studies (PROVE-IT or JUPITER) have shown that apart from any signs of glucose intolerance, some people develop diabetes under statin therapy. A meta-analysis of this work confirmed a 9% increase in the risk of diabetes. More importantly, it appears that in postmenopausal women, this increased risk can reach nearly 50%. In spite of this observation, it is not possible at the moment to dispense with statins because their cardiovascular benefits are such that there is no reason to question the benefit/risk ratio. Paradoxically, the trend is to intensify treatment because of a residual cardiovascular risk under statins.

With respect to ezetimibe, fenofibrates and niacin, beyond their respective association with statins, which has demonstrated lipid-lowering biological effects, there is limited data on glucose homeostasis.

Moreover, in diabetic and obese patients, white blood cells, circulating monocytes, macrophages and mature adipocytes have inflammatory characteristics. They have an impact on adipose tissue and other organs as they contribute to the development of disturbances in the liver, pancreas, muscles, brain or cardiovascular system. This inflammation is evidenced by the overexpression of biological biomarkers such as: TNFa, IL-6, adipokines, ACSL1, leukotrienes.

Tumor necrosis factor alpha (TNFα) plays a role in the development of insulin resistance associated with obesity. It also reduces the oxidation of fatty acids in hepatocytes and skeletal muscle. It inhibits adipocyte differentiation by acting on C/EBP and PPAR. It also increases lipolysis.

Interleukin-6 (IL-6) plays an important role in initiating and accelerating chronic inflammation. He is also involved in the resistance of insulin associated with obesity. The increase in these plasma levels is associated with variables (fasting plasma glucose level, LDL-c, total cholesterol, body mass index) that could contribute to the development of microvascular complications in diabetic patients.

On the other hand, PPARy (Peroxisome Proliferation Activated Gamma Receptor) attenuates the effect of TNFa and IL-6 in adipose tissue and thus improves insulin sensitivity. More generally, nuclear receptors, including PPARs, LXRs and RXRs mediate the regulation of cholesterol effluent from different cell types by activating transcription of ABCA1 and ABCG1 cholesterol transporters. They also increase the expression of the Niemann-Pick CI (NPC1) and C2 (NPC2) proteins involved in lysosome cholesterol trafficking. They are also involved in the downregulation of certain inflammatory genes (NF-KappaB, STAT, AP-1) by a transrepression mechanism.

Almost all adipokines are involved in inflammation related to the increase in fat mass and plays a role in the development of insulin resistance. Their high blood concentrations indicate that they also play a vital role in the establishment and development of a number of obesity related complications (including diabetes and cardiovascular disease). It should also be noted that the inflammation associated with diabetic atherosclerosis is correlated with the overproduction of ACSL1.

Leukotrienes produced by the fat cells of obese people promote inflammation and insulin resistance, the first step towards diabetes.

Furthermore, it is known that d-limonene possesses anti-diabetic and hypolipidemic properties, and can for this purpose be considered as a potential agent for preventing and treating metabolic disorders. Its antioxidant, anti-inflammatory and anticancer properties are known. In humans, d-limonene has demonstrated low toxicity after a single, repeated dose for one year. It can also dissolve gallstones containing cholesterol. It has a neutralizing effect on stomach acid and on the support of normal peristalsis, therefore relieves heartburn and gastroesophageal reflux disease (GERD).

Jing L et al., (2013) Eur J Pharmacol 2013 September 5; 715 (1-3): 46-55. Doi: 10.1016/j.ejphar.2013.06.022.Epub 2013 Jul. 6, determined the preventive and therapeutic effects of d-limonene on metabolic disorders in mice with high fat content induced by obesity. In pre-treatment, d-limonene decreased the size of white and brown adipocytes, lowered serum triglycerides (TG) and fasting blood glucose levels, and prevented hepatic lipid accumulation in C57BL/6 mice. fed with high fat foods. In therapeutic therapy, d-limonene reduced serum TG, low-density lipoprotein cholesterol (LDL-c), fasting blood glucose levels, glucose tolerance, and increased high-density lipoprotein cholesterol (HDL-c) in obese mice.

In addition, it is also known that lupeol (also known as agar-sterol or Clerodol) is a pharmacologically active compound with anti-inflammatory properties. Its antidiabetic and antioxidant properties are known. It is also known to provide substantial protection against abnormalities that manifest themselves in the early stage of hypercholesterolemic atherogenesis. Its anticancer properties are known. It should be noted that lupeol at the effective therapeutic dose shows no toxicity to normal cells and tissues.

The anti-diabetic and anti-oxidant potential of lupeol has been studied by Gupta R et al., [Nat Prod Res. 2012; 26 (12): 1125-9. Doi: 10.1080/14786419.2011.560845.Epub 2011 Nov. 1]. In their study, lupeol suppressed the progression of diabetes after 21 days, resulting in a decrease in glycated hemoglobin, serum glucose, and nitric oxide, with a concomitant increase in serum insulin levels. In addition, lupeol also increased antioxidant levels, with a decrease in the content of thiobarbituric acid-reactive substances. Its cholesterol-lowering effect was demonstrated in a study carried out by

Keishi Hata et al., (2008) [Article in Phytochemistry Letters1(4): 191-194.December 2008 DOI: 10.1016/j.phyto1.2008.09.007. In this study, lupeol strongly blocked TG synthesis and lipid droplet accumulation in 3T3-L1 cells stimulated by differentiation inducers. It is also known that beta-sitosterol inhibits the intestinal absorption of cholesterol and reduces cholesterol levels in the blood. Beta-sitosterol is also known to decrease systemic inflammation and increase immunity. Its anticancer properties are known. Its anti-diabetic and antioxidant potential are also known and referenced. Ivorra M D et al., [Pharmazie. 1990 April; 45 (4): 271-3] suggested in their study that beta-sitosterol-3-beta-D-glucoside exerts its action on intact pancreatic beta cells by stimulating insulin secretion.

In addition, Methyl Hydroxy Chalcone Polymer (MHCP) is known as an insulin mimetic. It seems to work in synergy with insulin. It is also known to reduce the resistance of fat cells to insulin, and thereby improves glucose metabolism.

Cinnamaldehyde is known for its hypoglycemic properties, and contributes to this effect to inhibit oxidative stress. Cinnamaldehyde, like epicatechin, is known to inhibit aggregates of tau and beta amyloid, which are characteristic of the brains of patients with Alzheimer's disease. It is also known for its hypolipidemic and anticancer properties.

### Technical Problem to Solve

An object of the present invention is to provide a novel pharmaceutical composition that can be used as a medicament and more particularly that it can be used in the treatment of concomitant or diabetic dyslipidemias, neurodegenerative diseases, cancers, bacterial, viral, fungal or parasitic infections.

Another object of the invention is to provide a novel pharmaceutical composition that can be used as a medicament and more particularly that can be used in the treatment of diseases that remedy all or some of the disadvantages related to the compositions of the aforementioned prior art. Another object of the present invention is to provide a pharmaceutical composition that can be used as a medicament, particularly for the therapeutic treatment of diabetes, hypercholesterolemia, hypertriglyceridemia and obesity.

Another object of the invention is to provide a pharmaceutical composition which makes it possible to inhibit the inflammatory phenotype of circulating monocytes, macrophages, white blood cells, pancreatic cells in diabetics.

Another object of the present invention is to provide a pharmaceutical composition which makes it possible to protect pancreatic cells secreting insulin from apoptosis and/or to reduce insulin resistance and hyperinsulinemia.

Another object of the present invention is to provide a pharmaceutical composition that can be used as a medicament in neurodegenerative diseases, in particular in the prevention of the formation of senile plaques, tissue lesions related to oxidative stress, characteristics common to diabetes and to the disease. Alzheimer.

Another object of the present invention is to provide a pharmaceutical composition which will decrease or even inhibit the affinity, the fusion between the pathogens (viruses, bacteria, parasites, etc.) and their target cells.

Another object of the present invention is to provide a pharmaceutical composition which makes it possible to inhibit the infectivity of cells, especially T cells, by HIV and its various variants, to inhibit viral replication, the senescence of immune cells and of activate a competent immune response. Another object of the present invention is to propose a composition pharmaceutical, especially as mentioned above, which has reduced toxicity and/or is well tolerated by patients.

### BRIEF DESCRIPTION OF THE INVENTION

To solve at least one of the above-mentioned technical problems, the present invention provides a pharmaceutical composition, which typically comprises, as active ingredient, a combination of d-limonene, lupeol, and a pharmaceutical agent. active agent selected from polymeric methylhydroxychalcone (MHCP), cinnamaldehyde, beta-sitosterol, curcumin, epicatechin and mixtures thereof.

The Applicant has indeed found that such a pharmaceutical composition proved to be active in dyslipidemias, lipodystrophies, metabolic diseases, autoimmune diseases, neurodegenerative diseases, in viral, bacterial, fungal, parasitic, and the cancer.

The Applicant has also demonstrated a synergistic effect of at least three of the constituents which provides a reinforced action of the composition of the invention on diseases referenced in the prior art, characterized by an increase in LDL-cholesterol, an elevation triglycerides, decreased HDL-cholesterol, hyperglycemia, insulin resistance, increased oxidative stress, senile plaque formation, overexpression of certain lipids and membrane proteins (glycosphingolipids and CXCR4), penetration of pathogens into their cells targets, chronic inflammation and systemic immune hyperactivation.

The Applicant has also found that the composition according to the invention has a synergistic effect on the regulation of PPARs, LXRs and RXRs.

### DETAILED DESCRIPTION

The pharmaceutical composition according to the invention can be used as a medicament, and in particular for its use in the preventive and curative treatment of concomitant or consecutive dyslipidemias to a diabetic or viral pathology, in the treatment of cancers, and in particular those associated with adipose tissue, in the treatment of neurodegenerative diseases, autoimmune diseases, infectious diseases, and especially HIV infection. According to a particular embodiment of the present invention, the composition of the invention may further comprise a mixture of beta-sitosterol and methylhydroxychalcone polymer (MHCP) or a beta-sitosterol and cinnamaldehyde mixture or a mixture of methylhydroxychalcone polymer (MHCP) and cinnamaldehyde or a mixture of beta-sitosterol, cinnamaldehyde and/or epicatechin or a mixture of cinnamaldehyde with one of the epicatechin derivatives.

Preferably, the composition does not include a methylhydroxychalcone polymer, cinnamaldehyde and beta-sitosterol, or a mixture of epicatechin and its derivatives.

By way of example, it may comprise, as a mass percentage of the total mass of the active ingredients, a mass percentage of d-limonene substantially equal to or greater than 10% and substantially equal to or less than 55, and in particular substantially equal to or greater than 20%) and substantially equal to or less than 40%, a percentage of lupeol substantially equal to or greater than 15%>and substantially equal to or less than 55%, and in particular substantially equal to or greater than 30% and substantially equal to or less than 40%, a percentage of cinnamaldehyde substantially equal to or greater than 15% and substantially equal to or less than 45%, and especially substantially equal to or greater than 20% and substantially equal to or less than 40%, a percentage of MHCP substantially equal to or greater than 15% and substantially equal to or less than 40%, and in particular substantially equal to or greater than 25% and substantially equal to or less than 35%, a percentage of beta-sitosterol when the said composition contains this ingredient, substantially equal to or greater than 10% and substantially equal to or less than 45%, and in particular substantially equal to or greater than 15% and substantially equal to or less than 30%.

When the composition comprises MHCP or epicatechin and cinnamaldehyde their respective percentage by weight relative to the total mass of the active ingredients is in particular equal and in particular substantially equal to 15%. The composition according to the invention further comprises at least one pharmaceutically acceptable excipient. This excipient can be solid or liquid. It may be chosen, for example, from purified water, ethyl alcohol, propylene glycol, glycerin, vegetable oils, animal oils, hydrocarbons, silicones, sugars such as glucose, levulose, wheat starch, corn starch, potato starch, xanthan gum, gum arabic, gum tragacanth, gum Sterculia, guar gum or “guaranates”, pectins, alginates, carrageenates, agar or Agar-Agar, gelatin, cellulose and its derivatives.

The composition of the invention may be administered by any suitable route, for example by oral, rectal, local (topical, for example), intraperitoneal, systemic, intravenous, intramuscular, subcutaneous or mucosal, especially sublingual or using a patch, or in encapsulated form, or immobilized on liposomes, microparticles, microcapsules, associated with nanoparticles and the like. Mention may be made, by way of non-limiting examples of excipients suitable for oral administration, talc, lactose, starch and its derivatives, cellulose and its derivatives, polyethylene glycols, polymers of acrylic acid, gelatin, magnesium stearate, animal, vegetable or synthetic fats, paraffin derivatives, glycols, stabilizers, preservatives, antioxidants, wetting agents, anti-caking agents, dispersants, emulsifiers, taste modifying agents, penetrating agents, solubilizing agents. The techniques of formulation and administration of drugs and pharmaceutical compositions are well known in the art here considered, the skilled person may in particular refer to the book Remington's Pharmaceutical Sciences, latest edition.

According to the invention, the composition can advantageously be administered orally by intravenous injection.

Advantageously, the composition according to the invention is adapted to be administered orally or intravenously at a dose equal to or greater than 40 mg/kg/24 h and equal to or less than 200 mg/kg/24 h in one or more doses to a mammal presenting such a need.

By way of examples, the composition of the invention may be used in the preventive and/or curative treatment of dyslipidemias, insulin resistance, iatrogenic hyperlipidemias, especially in HIV-infected patients treated with antiretroviral combinations, in the preventive and/or curative treatment of atherosclerosis, coronary heart disease selected from angina pectoris or myocardial infarction, carotid artery disease, especially stroke and aneurysm brain, peripheral arterial disease, pulmonary embolism. The composition according to the invention can advantageously be used in patients suffering from a chronic inflammatory disease and/or resulting from an infection caused by least a pathogen and/or systemic immune hyperactivation and/or lipid imbalance and/or malfunction of the cholesterol cell transporter, particularly in chronic inflammatory disease of diabetes-related tissues, obesity, AIDS, Crohn's disease, hepatocellular insufficiency, hepatic steatosis, cholecystitis, vesicular lithiasis, in autoimmune diseases, including type 1 diabetes, autoimmune thyroiditis, autoimmune thyroiditis, Immune, autoimmune uveitis and autoimmune retinitis, Sjogren's syndrome, systemic lupus erythematosus, multiple sclerosis, rheumatoid arthritis, scleroderma, polymyositis and mixed connectivity in neurodegenerative diseases in this case, Alzheimer's disease, Parkinson's disease, multiple sclerosis, amyotrophic lateral sclerosis or charcot disease, dementia vascu in glomerular nephropathies, in cancers, especially those associated with adipose tissue.

In the case of AIDS treatment, the Applicant has demonstrated that the composition according to the invention gave good results at least in vitro and showed very low toxicity for healthy cells of the liver.

The mode of action of the composition of the invention is not fully understood. It is more than likely that it acts simultaneously on different mechanisms responsible for diseases cited in the prior art. It would exert its action by a synergistic regulatory effect on nuclear receptors, in particular PPARs, LXRs and RXRs, thus allowing the efflux of cellular cholesterol, the inhibition of the production of pro-inflammatory cytokines, the inhibition of formation and/or overexpression and/or dysfunction of certain sphingolipids, including glycosphingolipids (such as globotriaosylceramide, monosialogangloside), membrane proteins, including LRP1, T cell receptor (TCR), CXCR4. It would also inhibit the chemokine protein SDF-1, a cognate ligand of CXCR4, and consequently the formation of the CXCR4/SDF-1 axis which plays a fundamental role in establishing an immunosuppressive microenvironment, forming a new blood supply, tumor growth and metastasis.

Thus, the composition according to the invention allows the destructuration, the restructuring of the lipid composition of the cells, and in particular membrane lipid rafts, targets of bacteria, parasitic protozoa and viruses, and thus prevents the stabilization of these microorganisms. areas, setting up a fusion complex, training of synapse, endocytosis, and thus ultimately the penetration of these pathogens into the cells.

In addition, the modification of the lipid composition of these membrane micro-domains causes a conformational change, an alteration of the functional or dysfunctional activity of the proteins found therein, in particular the G-protein coupled receptors, and ultimately a alteration of cellular signaling pathways involved in many pathophysiological processes, including infections caused by pathogens, in acquired immunodeficiency syndrome, cancer, obesity, metabolic diseases, autoimmune diseases and diseases neurodegenerative.

This lipid disorganization could also be done in the envelope of infectious agents, and then modify the conformation of the information proteins involved in the infection process, their binding to their target cells. This mechanism would apply to viruses, bacteria with biophysical and biochemical properties similar to their target cells, particularly in the lipid bilayer envelope.

The composition according to the invention may advantageously be used to reduce or inhibit the infectivity of pathogens, the inflammatory phenotype of cells, oxidative stress, the senescence of immune cells, and to increase an innate and adaptive immune response. Consequently, the composition according to the invention can be used in the treatment of infections caused by pathogens and their various variants, in particular retroviruses (lentiviruses including HIV-1 and HIV-2, oncoviruses, and spumaviruses), measles virus, influenza virus, smallpox virus, yellow fever virus, West Nile virus, vesicular stomatitis virus (VSV), hepatitis B virus (HBV), hepatitis C virus (HCV), cytomegalovirus (GVIV), Epstein-Barr virus (EBV), human herpesvirus 8 (HHV8), Ebola virus, some rotavirus, some naked viruses, in the treatment of bacterial infections, including Escherichia coli infection, Mycobacterium tuberculosis, treatment of Plasmodium falciparum infection, and in the treatment of cancers. These cancers include AIDS-related cancers, including sarcoma

Kaposi, burkitt lymphoma, immunoblastic lymphoma, brain lymphoma primitive, non-Hodgkin's lymphoma (NHLH), cervical cancer, and non-classifying AIDS cancers selected from oral cancer, stomach cancer, colon cancer, especially cancer invasive colon or colorectal, rectal cancer, anal cancer, liver cancer, hepatocellular carcinoma, gallbladder cancer, pancreatic cancer, lung cancer, especially lung adenocarcinoma, leukemia in chronic or acute form, multiple myeloma, Hodgkin's lymphoma, brain tumors and others with localization in the nervous system, bladder cancer, ovarian cancer, cancer of the uterus, testicular cancer, kidney cancer, prostate cancer and breast cancer, especially those associated with adipose tissue, bone tumors.

The present invention also relates to a pharmaceutical preparation which comprises the composition according to the invention, and, in addition, as a mixture or separately packaged, at least one antidiabetic agent and/or a lipid-lowering agent and/or an anti-infectious agent and/or an anti-cancer agent for their use in the therapeutic treatment of diabetes, dyslipidemia, obesity, atherosclerosis, cardiovascular diseases, infections caused by pathogens, cancers, and especially those associated with adipose tissue, simultaneously, sequenced or spaced in time.

By way of example, the anti-diabetic agent may be chosen from the biguanides, the sulphonylureas and glinides, the alpha-glucosidase inhibitors, the incretins, including GLP-1, insulin and the hypolipidemic agent. may be chosen from statins, fibrates, Ezetimibe, nicotinic acid, cholestyramine, the anti-infective agent may be chosen from antiretrovirals, in particular nucleoside or non-nucleoside reverse transcriptase inhibitors, inhibitors of protease, fusion inhibitors and integrase inhibitors, antibiotics, antiparasitic agents, antimycotics, the anticancer agent may be chosen from the anti-metabolites (methotrexate, capecitabine, 5-fluorouracil), the alkylating agents (cisplatin, mitomycin c, busulfan) and the relatives (melphalan, chloraminophene, cyclophosphamide), the molecules having an action on the mitotic spindle (vinlastine, vincristine, doxetaxel), I tyrosine kinase inhibitors (afatinib, erlotinib, sunitinib), threonine kinase inhibitors (vermurafenib, everolimus, temsirolimus), agents acting on topoisomerase (daunorubicin, doxorubicin, etoposide), proteasome inhibitors, inhibitors of DNA methyltransferase, histone deacetylase inhibitors, immunomodulators (the interferons, corticosteroids, talimogen), monoclonal antibodies (cetuximab, gemtuzumab, trastuzumab, bevacizumab, rituxumab), certain genetically modified viruses that preferentially target cancer cells, glutathione, vitamin C, calcium folinate and mixtures thereof, and in particular the mixture of two of said anticancer agents, the radioactive agents which can be used in brachytherapy and/or the injectable or ingestible active metabolites.

The present invention also relates to a pharmaceutical preparation which comprises in combination d-limonene, lupeol and/or beta-sitosterol, methylhydroxychalcone polymer and/or cinnamaldehyde and optionally curcumin, epicatechin.

The present invention also relates to a dietary supplement which comprises in combination d-limonene, lupeol and a pharmaceutically active agent chosen from methylhydroxychalcone polymer, cinnamaldehyde, beta-sitosterol and mixtures thereof, and optionally curcumin, epicatechin.

### Definitions

The term “therapeutic treatment” refers to curative treatment and prophylactic treatment; within the meaning of the present invention, a therapeutic treatment makes it possible to at least partially restore, at least partially correct or at least partially modify physiological functions by exerting a pharmacological, immunological or metabolic action.

The term “patient” refers to an animal or human mammal. The composition according to the invention can also be used in veterinary medicine.

The term “patients with diabetes” refers to patients with type 1 diabetes, patients with type 2 diabetes, patients with gestational diabetes, patients with diabetes insipidus and patients with renal diabetes.

The term “dyslipidemia” refers to hyperlipidemia and hypolipidemia determined according to the criteria in force.

The term “atherosclerosis” refers to the loss of elasticity of the arteries, due to sclerosis caused by accumulation of fat (lipids, mainly LDL cholesterol), in one of the three tunnels constituting the wall of the arteries (intimal).), and interesting before everything, the big and medium arteries.

The term “inflammation” refers to a set of reactions generated by the body in response to aggression. This can be of external origin as a wound, an infection, a trauma, or coming from within the body itself as in autoimmune pathologies.

The term “hepatocellular insufficiency” refers to the clinical and biological manifestations secondary to the alterations of the hepatocellular functions, in particular in the synthesis, purification, biliary secretion.

For the purpose of the present invention, an “anti-cancer agent” is an element that has, at least in vitro, an action against cancer cells, regardless of its mechanism of action. For the purposes of the present invention, the term “action” means the destruction or at least partial modification of the cancer cells, which makes it possible in particular to limit the proliferation of the cancer cells and/or their propagation.

The term “infection” refers to the invasion of a living organism by germs, more specifically pathogenic microorganisms such as a bacterium or a virus requiring a host, most often a cell whose constituents it uses to multiply.

For the purposes of the present invention, a “food supplement” is a foodstuff whose purpose is to supplement the normal diet and which constitutes a concentrated source of nutrients or other substances having a nutritional or physiological effect alone or in combination.

With respect to the anti-diabetic and/or lipid-lowering and/or anti-cancer agents mentioned, the terms used, unless otherwise indicated, include the constituent isomers, the stereoisomers of conformation, the enantiomers and the diastereoisomers of the chemical compound in question. With regard to cinnamaldehyde (CA) in the composition according to the invention, the term includes, unless otherwise indicated, its derivatives, in particular 2-hydroxycinnamaldehyde (HCA), 2′-benzoyloxycinnalmaldehyde (BCA), formation dimers, in particular HCA-HCA, BCA-BCA, CA-CA.

As regards the epicatechin in the composition according to the invention, the term includes, unless otherwise indicated, its derivatives, in particular catechin, gallocatechin (GC), epicatechin gallate (ECG), epigallocatechin (EGC), epigallocatechin gallate (EGCG). [Examples]

The percentage of the compositions below is a percentage by weight relative to the total mass of the active ingredients. Composition: d-limonene (40%), lupeol (30%) and MHCP (30%).

Composition Ib: d-limonene (40%), lupeol (30%) and cinnamaldehyde (30%)

Composition 2: d-limonene (40%), lupeol (30%), MHCP (15%) and cinnamaldehyde (15%).

Composition 3a: d-limonene (30%), lupeol (30%), and MHCP (40%).

Composition 3b: d-limonene (30%), lupeol (30%), and cinnamaldehyde (40%). Composition 4a: d-limonene (15%), lupeol (50%), beta-sitosterol (10%) and MHCP (25%).

Composition 4b: d-limonene (15%), lupeol (50%), beta-sitosterol (10%) and cinnamaldehyde (25%).

Composition 5a: d-limonene (20%), lupeol (20%), beta-sitosterol (40%) and MHCP (20%).

Composition 5b: d-limonene (20%), lupeol (20%), beta-sitosterol (40%) and cinnamaldehyde (20%).

Composition 6a: d-limonene (25%), lupeol (35%), beta-sitosterol (15%) and MHCP (25%). Composition 6b: d-limonene (25%), lupeol (35%), beta-sitosterol (15%) and cinnamaldehyde (25%).

Composition 7a: d-limonene (50%), lupeol (15%), beta-sitosterol (10%) and MHCP (25%). Composition 7b: d-limonene (50%), lupeol (15%), beta-sitosterol (10%) and cinnamaldehyde (25%).

### Experimental Results

Different cells were studied: 3T3-L1 cells, monocytes, macrophages, white blood cells, hepatocytes, adipocytes. They have been selected based on their ability to accumulate lipid droplets. These cells, especially white blood cells, have inflammatory characteristics in diabetics.

The cells were maintained in DMEM, supplemented with 10% fetal bovine serum (FBS) and 1% antimycotic antibiotic solution (MPS), containing penicillin, streptomycin, and amphotericin B under standard growth conditions. (5% CO 2, 37° C., humidified atmosphere). The above compositions were dissolved and diluted in DMSO. The above cells were treated with solutions (10-80 μM) for 48 h in complete cell media. All treatment and control protocols were prepared as previously described. The aforementioned solutions have resulted in selective inhibition of differentiation of 3T3-L1 cells into adipocytes even in the presence of an additional prodifferential agent such as rosiglitazone. The effect of this differentiation was quantified by Oil Red O staining: Changes in the expression of genes and proteins associated with differentiation and adipogenesis were observed. The aforementioned solutions resulted in a significant inhibition of the accumulation of lipid droplets in monocytes, macrophages, white blood cells, hepatocytes, regulated the expression of the peroxisome proliferation-activated gamma receptor (PPARy) resulting in a decreased proinflammatory cytokines, inhibited the formation, overexpression and/or dysfunction of certain membrane lipids (sphingolipids, especially glycosphingolipids), certain membrane proteins as a function of concentration. They also decreased the affinity of pathogens for their target cells.

These results taken as a whole suggest that this pharmaceutical composition inhibits the manufacture of adipose tissue and thereby reduces insulin resistance. Extrapolation can be done to visceral adipose tissue (TAV) or subcutaneous adipose tissue (TA se) which plays a central role in the pathophysiology of Metabolic Syndrome. Moreover, this pharmaceutical composition could inhibit tumor progression and resistance to anticancer treatments by inhibiting the secretion of cancer-associated adipocytes (CAA), especially proinflammatory cytokines (such as TNFα, ILip, IL6, IL8), pro-angiogenic molecules (such as VEGF for Vascular Endothelial Growth Factor), chemokines (like MCP-1 for monocyte chemoattracting protein-1, S1P for sphingosine-1-phosphate), growth factors (such as HGF, for Hepatocyte Growth Factor), extracellular matrix proteins and its remodeling as well as the release of free fatty acids. It decreases or even inhibits the infectivity of pathogens and their different variants. Therefore, this pharmaceutical composition can be used rightly in the preventive and curative treatment of obesity, diabetes, dyslipidemias and their consequences, neurodegenerative diseases, infections caused by pathogens, cancers, especially those associated to adipose tissue, such as breast cancer, prostate cancer.

1. The pharmaceutical composition, which is characterized, it comprises in combination d-imoriene, lupeol and/or beta-sitosterol, cinnamaldehyde and/or methylhydroxychalcone polymer and as an option as curcumin, epicatechine.

2. The pharmaceutical composition in accordance with the claim 1, which has been characterized, further comprises a beta-sitosterol and cinnamaldehyde mixture or a mixture of beta-sitosterol and methylhydroxychalcone polymer (MHCP) or a mixture of methylhydroxychalcone polymer (MHCP) and cinnamaldehyde or a mixture of cinnamaldehyde and/or epicatechine and/or curcumin.

3. The pharmaceutical composition, in accordance with claim 1, which have been characterized and comprised as a mass percentage of the total mass of the active ingredients, a mass percentage of d-limonene being substantially equal to or greater than 10% and substantially equal to or less than 55%, and in particular, being substantially equal to or greater than 20% and being substantially equal to or less than 40%, a percentage of lupéol being substantially equal to or greater than 15% and being substantially equal to or less than 55%, and in particular being substantially equal to or greater than 30% and being substantially equal to or less than 40%, a percentage of cinnamald\tilde{e}hyde being substantially equal to or greater than 15% and being substantially equal to or less than 45%, and especially being substantially equal to or greater than 20% and being substantially equal to or less than 40%, a percentage of MHCP being substantially equal to or greater than 15% and being substantially equal to or less than 40%, and especially being substantially equal to or greater than 25% and being substantially equal to or less than less than 35%, a percentage of beta-sitosterol when said composition contains this ingredient, being substantially equal to or greater than 10% and being substantially equal to or less than 45%, and especially being substantially equal to or greater than 15% and being substantially equal to or less than 30%.

4. The pharmaceutical composition, in accordance with claim 1, for its usage in the prevention and/or curative treatment of dyslipidernias, insulin-resistance, iatrogenic hyperlipidemia, more particularly, in patients who are infected with HIV and who have been treated with antiretroviral combinations, clans. The prevention and/or curative treatment of atherosclerosis, coronary heart disease selected from angina pectoris or myocardial infarction, carotid artery disease, in particular, (missing) cerebrovascular accident and cerebral aneurysm, peripheral arterial disease, pulmonary embolism.

5. The pharmaceutical composition, in accordance with claim 1, for their usage in the treatment of chronic inflammatory disease and/or resulting from infection caused by at least one pathogenic agent and/or systemic immune hyperactivation and/or lipid imbalance and/or cholesterol cell transporter dysfunction, more particularly, in chronic inflammatory disease of diabetes, obesity, AIDS, Crohn's disease, hepatocellular insufficiency, Hepatitis steatosis, cholecystitis, vesicular lithiasis, in autoimmune diseases, including type 1 diabetes, autoimmune thyroiditis, autoimmune hepatopathies, autoimmune uveitis and autoimmune retinitis, Gougerot-Sjögren, lupus erythematous disseminin, multiple sclerosis, rheumatoid arthritis, scleroderma, polymyositis and mixed connective tissue disease, in neurodegenerative diseases, in this case, Alzheimer's disease, Parkinson's disease, multiple sclerosis, amyotrophic lateral sclerosis or charcot disease, vascular dementia, in glomerular nephropathies, in cancers, more particularly, those associated with adipose tissue.

6. The pharmaceutical composition, in accordance with claim 1, which have been characterized in this carry out destructuring and restructuring of the lipid composition of cells, infectious agents, in particular, the membrane, amends the conformation of the proteins found there, and alter, thus preventing the stabilization of membrane micro-domains, the penetration of pathogens into cells, the cell signaling pathways involved in many pathophysiological processes, particularly in infections, acquired immunodeficiency syndrome, inflammation, cancer, obesity, metabolic diseases, autoimmune diseases and neurodegenerative diseases,

7. The pharmaceutical composition, in accordance with claim 6, for its usage in the treatment of infections caused by pathogens and their different variants, in particular, the retroviruses (lentiviruses including HIV-1 and HIV-2, oncoviruses, and spumaviruses), the virus measles, influenza virus, smallpox virus, yellow fever virus, West Nile virus, vesicular stomatitis virus (VSV), hepatitis B virus (HBV), hepatitis C virus (HCV), cytomegalovirus (CMV), Ebola virus, certain rotaviruses, for treatment of Escherichia coli, Mycobacterium tuberculosis, Plasmodium falciparum, and in the treatment of cancers, including AIDS-related cancers selected from Kaposi's sarcoma, burkitt lymphoma, immunoblastic lymphoma, primary brain lymphoma, non-Hodgkin's lymphoma (NHL), cervical cancer, and non-class cancers AIDS selected among the mouth cancer, stomach cancer, colon cancer, especially invasive colon or colorectal cancer, rectal cancer, anal cancer, liver cancer, hepatocellular carcinoma, cancer of the vesicle biliary, pancreatic cancer, lung cancer, especially lung adenocarcinoma, leukemia in chronic or acute form, multiple myeloma, Hodgkin's lymphoma, encephalic tumors, and others, viz., nervous system location, bladder cancer, ovarian cancer, uterine cancer, testicular cancer, kidney cancer, prostate cancer and breast cancer, in particular, those associated with adipose tissue, bone tumors.

8. The pharmaceutical preparation, as characterized, comprises the composition according to claim 1 and, in addition, separately a mixture or conditioned at least one antidiabetic agent and/or a hypolipemic agent and/or an anti-infectious agent and/or an anti-cancer agent for their usage in the therapeutic treatment of diabetes, dyslipidemia, obesity, atherosclerosis, cardiovascular diseases, infections caused by pathogens, cancers, especially those associated with adipose tissue, simultaneous, sequential, or spaced manner in reasonable duration.

9. The pharmaceutical preparation, in accordance with claim 8, which has been characterized, as antidiabetic agent is selected from biguanides, hypoglycemic sulphonamides and glinides, alpha-glucosidase inhibitors, incretins including GLP-1, insulin hypolipemic agent is selected from statins, fibrates, Ezetimibe, nicotinic acid, cholestyramine, the antiviral agent is selected from nucleoside or non-nucleoside reverse transcriptase inhibitors, protease inhibitors, inhibitors of fusion and integrase inhibitors, the anti-cancer agent is selected from the anti-metabolites (methotrexate, capecitabine, 5-fluorouracil), the alkylating agents (cisplatin, mitomycin c, busulfan) and the apparent ones (melphalan, chloraminophene, cyclophosphamide), molecules with action on the mitotic spindle (vinlastine, vincristine, doxetaxel), tyrosine kinase inhibitors (afatinib, erlotinib, sunitinib), inhibitors threonine kinase (vermurafenib, everolimus, temsirolimus), agents acting on topoisomerase (daunorubicin, doxorubicin, etoposide), proteasome inhibitors, inhibitors of DNA methyltransferase, histone deacetylase inhibitors, immunomodulators (interferons, corticosteroids, talimogene), monoclonal anti-bodies (cetuximab, gemtuzumab, trastuzumab, bevacizumab, rituxumab), certain genetically modified viruses which, as an preference, targets cancer cells, glutathione, vitamin C, calcium folinate and their mixtures, and in particular the mixture of two of said anticancer agents, the radioactive agents that can be used in curietherapie and/or the injectable or ingestablessimultaneous, sequenced, or space-active metabolites in time.
